(19) United States
(12) Patent Application Publication
(10) Pub. No.: US 2024/0366887 A1
(43) Pub. Date: Nov. 7, 2024

(54) Device & method for preventing wrong/unintentional/unauthorized injections

(71) Applicant: Javad Hajizadeh Rineh, Tehran (IR)

(72) Inventor: Javad Hajizadeh Rineh, Tehran (IR)

(21) Appl. No.: 18/688,781

(22) PCT Filed: Sep. 4, 2021
(86) PCT No.: PCT/IB2021/058080

§ 371 Date: Mar. 3, 2024

(51) Int. Cl. A61M 5/50; A61M 39/10

(52) U.S. Cl. CPC A61M 5/5086; A61M 39/10; A61M 2205/14; A61M 2205/18; A61M 2205/44; A61M 2205/582; A61M 2205/583

(57) ABSTRACT

A device for preventing wrong route/unintentional/unauthorized injection into catheters and connectors for intravascular, hypodermic, and neuraxial applications includes a female end (connector) connected to a male end (connector), and a detecting mechanism for detecting the insertion of an external male end (connector) such as a syringe into the female end of the device. The device may further include an input/output (I/O) interface coupled to the detecting mechanism. The detecting mechanism is further configured to urge the I/O interface to generate an alarm responsive to the detection of the insertion of a male connector into the female end of the device. By connecting this device to any female ends in which we need an extra layer of security we can protect that female connection from possible errors. This device is applicable in any situations in which we need more caution such as preventing wrong route drug injections.

### TECHNICAL FIELD

The present disclosure generally relates to the catheters and connectors of medical devices and particularly relates to the catheters and connectors for intravascular, hypodermic, and neuraxial applications. More particularly, the present disclosure relates to methods, systems, and devices for preventing wrong route/unintentional/unauthorized injection into venous/epidural/arterial lines.

### BACKGROUND

Catheters may be inserted into a patient's body for various purposes. Peripheral venous catheters or central venous catheters may be utilized to administer intravenous fluids, medication, or parenteral nutrition. Intra-arterial catheters may be utilized for sampling or direct measurement of blood pressure in an artery. An epidural catheter may be utilized for the administration of local anesthetics and narcotics medication into the epidural space.

The abovementioned catheters may be associated with other connections such as stopcocks, adapters, and ports that may have similar structures and shapes. Such connections usually have female ends (connectors) and are generally designed to be adaptable with various types of male ends (connectors) such as syringes and sets. Consequently, unintentional or wrong injections into such ports is an ever-present risk. Unintentional, wrong or unauthorized injections into arterial, epidural or venous lines may have serious consequences for the patient.

Intra-arterial catheters are generally utilized in intra-arterial cannulation procedures to provide access to an artery system of a patient. Intra-arterial catheters are most commonly utilized in intensive care medicine and anesthesia to allow for direct real-time monitoring of blood pressure and to obtain samples for arterial blood gas analysis. Such arterial lines are not generally utilized for administering medication and accidental injection of a drug into an arterial line may potentially lead to adverse consequences, such as tissue necrosis and even limb loss. Acute manifestations, as well as chronic manifestations may be expected with an unintentional intra-arterial injection. Current approaches to prevent the unintentional intra-arterial injection may include utilizing only catheters and tubing without injection ports for arterial pressure transduction; utilizing clear labeling and color-coding lines, connections and sets; keeping a port, if necessary, close to the insertion site of the catheter; tracing each extension to the site of the catheter before drug injections; utilizing one-way catheters and connections; and training all healthcare professionals who are involved in drug administrations about the risks and complications of unintentional intra-arterial injections.

An epidural catheter may allow access to the epidural space to inject medication, such as local anesthetics or narcotics for alleviation of pain. One common application of epidural catheters is in a labor procedure to control pain during childbirth. Unintentional/wrong injections into an epidural line may occur when the epidural line is mistaken for a venous line and unauthorized medications for regional anesthesia may be injected into the epidural line, especially by less-skilled medical practitioners. Such unintentional injections into the epidural line are undesirable and may be harmful to the patient.

Another form of undesirable injection into a body cavity is an unauthorized injection into venous/epidural/arterial lines. Such an unauthorized injection is especially a serious concern regarding patients with a substance abuse history or suicidal tendencies. Such patients may try to inject drugs into the venous/epidural/arterial lines, especially venous line without informing the caregivers, which may lead to unwanted drug interactions and in high doses it may even lead to death.

Despite all the measures that are being taken in hospitals and healthcare facilities, the risk of unintentional/wrong/unauthorized injections are still relatively high. Many patients in operation rooms and intensive care units (such as ICU and CCU) have multiple connections for various purposes. Other factors such as the need for urgent drug injections, high-stress situations, poor lighting environments, and fatigue practitioners can intensify the probability of wrong route drug injections.

There is, therefore, a need for a method, system, and device that may alarm the healthcare professionals when an injection is about to happen in any of the lines connected to the body cavities of a patient. There is further a need for a device that may be adaptable to all types of catheters and connectors for intravascular, hypodermic, and neuraxial applications, such as arterial, epidural, and venous catheter and all connected connectors to these lines and may be configured to prevent unintentional/wrong or unauthorized injections into the aforementioned lines.

### SUMMARY OF DISCLOSURE

This summary is intended to provide an overview of the subject matter of the present disclosure and is not intended to identify essential elements or key elements of the subject matter, nor is it intended to be used to determine the scope of the claimed implementations. The proper scope of the present disclosure may be ascertained from the claims set forth below in view of the detailed description and the drawings.

According to one or more exemplary embodiments, the present disclosure is directed to a device for preventing wrong route/unintentional/unauthorized injection into catheters and connectors for intravascular, hypodermic and neuraxial applications. An exemplary device may include a female end (connector) that may be configured to receive an external male end (connector), a male end (connector) that may be connected in fluid communication with an exemplary female end (connector). An exemplary female end (connector) may further include an annular wall that may encompass a fluid passage between an exemplary female end (connector) and an exemplary male end (connector).

In an exemplary embodiment, an exemplary device may further include a switch with an actuation lever that may be mounted on an exemplary annular wall. An exemplary annular wall may include a slit. An exemplary actuation lever may be configured to extend into an exemplary female connector via an exemplary slit in a fluid-tight manner.

In an exemplary embodiment, an exemplary device may further include a spring-loaded piston rod that may be coaxially mounted within an exemplary fluid passage, and a piston that may be coupled to an exemplary spring-loaded piston rod. An exemplary piston may be moveable along an exemplary longitudinal axis of an exemplary fluid passage towards an exemplary male end (connector) with an exemplary spring-loaded piston rod.

In an exemplary embodiment, an exemplary spring-loaded piston rod may be extended along an exemplary longitudinal axis of an exemplary fluid passage and an exemplary male end (connector) between a first end disposed within an exemplary female end (connector) and an opposing second end disposed within an exemplary male end (connector). An exemplary first end may be configured to provide a contact surface within an exemplary female end (connector). An exemplary spring-loaded piston rod may be configured to move along an exemplary longitudinal axis of an exemplary fluid passage towards an exemplary male end (connector) in response to an exemplary external male end (connector) being inserted into an exemplary female end (connector) pressing against an exemplary contact surface.

In an exemplary embodiment, an exemplary piston may be configured to contact and press an exemplary actuation lever in response to an exemplary spring-loaded piston rod moving along an exemplary longitudinal axis of an exemplary fluid passage towards an exemplary male end (connector). An exemplary switch may be configured to generate a detection signal responsive to the mechanical trigger being pressed.

### BRIEF DESCRIPTION OF FIGURES

The novel features which are believed to be characteristic of the present disclosure, as to its structure, organization, use and method of operation, together with further objectives and advantages thereof, will be better understood from the following drawings in which a presently preferred embodiment of the present disclosure will now be illustrated by way of example. It is expressly understood, however, that the drawings are for illustration and description only and are not intended as a definition of the limits of the present disclosure. Embodiments of the present disclosure will now be described by way of example in association with the accompanying drawings in which:

FIG. 1 illustrates a block diagram of a system for preventing wrong route/unintentional/unauthorized injection into catheters and connectors for intravascular, hypodermic and neuraxial applications, consistent with one or more exemplary embodiments of the present disclosure;

FIG. 2A illustrates a sectional side view of a device, consistent with one or more exemplary embodiments of the present disclosure; and

FIG. 2B illustrates an exploded view of a device, consistent with one or more exemplary embodiments of the present disclosure.

### DETAILED DESCRIPTION

The novel features which are believed to be characteristic of the present disclosure, as to its structure, organization, use and method of operation, together with further objectives and advantages thereof, will be better understood from the following discussion.

According to one or more exemplary embodiments, the present disclosure is directed to a system, device and methods for preventing wrong route/unintentional/unauthorized injection into catheters and connectors for intravascular, hypodermic and neuraxial applications. An exemplary system and device may be connected to one of an arterial line, a venous line, or an epidural line. An exemplary device may include a detecting mechanism that may be configured to trigger an alarm in response to an external male end (connector), such as a syringe, being inserted into an exemplary female end (connector) of the device. An exemplary detecting mechanism may be coupled to an input/output (I/O) interface that may include an alarm that may be configured to produce a plurality of one or more audio, tactile or visual indications responsive to an exemplary detecting mechanism being activated by an external male end (connector), such as a syringe. An exemplary alarm may be configured to produce either a local alarm or a central alarm in the nursing station.

For example, in an operating room, each of arterial or epidural lines and all of the connected connectors to mentioned lines may be equipped with an exemplary device and an exemplary I/O interface coupled to each of exemplary devices may be configured to produce a local alarm whenever an external male end (connector), such as a syringe connect to the female end (connector) of the device and an injection is about to happen. Such alarms, which may be unique to each line, may help the healthcare professionals be alert whenever they want to perform an injection in a particular line. This way the risk of unintentional/wrong injections may be significantly lowered.

In another example, for a patient with suicidal tendencies or a history of substance abuse, where the patient themselves may probably inject unauthorizedly into a venous or epidural line, and maybe an arterial line, an alarm mechanism provided by an exemplary device connected to the lines may alarm the healthcare professionals and prevent such unauthorized injections. In this example, an exemplary I/O interface of an exemplary device may include a central alarm system located in a nursing station. This way the provided intravenous and epidural line connected to the patient may be under constant supervision and the chances of the patient being able to inject themselves unauthorizedly will be very low.

An exemplary device may include a female end (connector) that may provide an injection port and a male end (connector) that may be configured to be connected to a line, such as an arterial line, an epidural line, or a venous line. An exemplary female end (connector) and an exemplary male end (connector) may be in fluid communication and may form a fluid passage that may extend along and between an exemplary female end (connector) and male end (connector). An exemplary female end (connector) may be configured to be adapted with various types of external male connectors, such as lock, Luer lock, slip, Luer slip syringes (connectors). As used herein, an exemplary female end (connector) being in fluid communication with an exemplary male end (connector) may refer to a fluid being able to flow from an exemplary female end (connector) towards an exemplary male end (connector) through an exemplary fluid passage.

An exemplary device may further include a detection mechanism. An exemplary detection mechanism may be configured to be activated in response to an external male end (connector), such as a syringe being inserted into an exemplary female end (connector) of an exemplary device. Such activation of an exemplary detection mechanism may urge an exemplary alarm to go off and alert a healthcare professional that an external male end (connector) such as a syringe has been inserted into an exemplary female end (connector) of the device. Here, such alarm may help a healthcare professional to double check the port to prevent any possible injections into a wrong port.

An exemplary detection mechanism may utilize a sensor (switch) that may be one of a mechanical sensor (switch), an electronic sensor (switch), an optical sensor (switch), an ultrasonic sensor (switch), and a magnetic sensor (switch). An exemplary sensor (switch) may be configured to be triggered or activated in response to an external male end (connector) such as a syringe being inserted into an exemplary female end (connector) of the device. An exemplary sensor (switch) may be coupled to an exemplary alarm of an exemplary I/O interface and in response to an exemplary sensor (switch) being activated, an exemplary I/O interface may urge an exemplary alarm to produce an audio, tactile or visual indication.

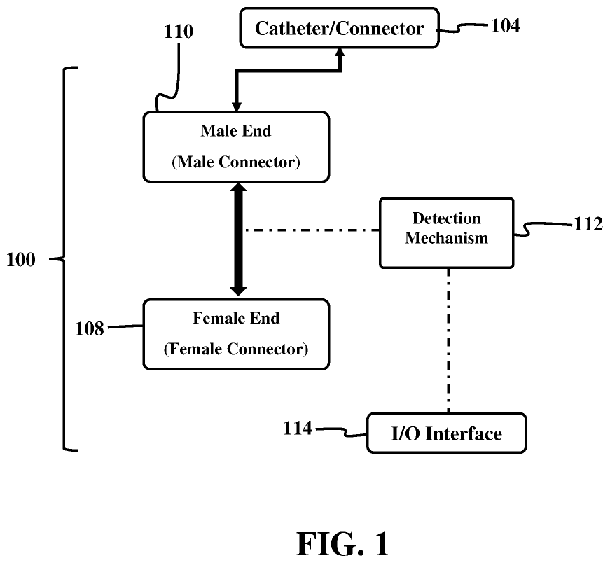

FIG. 1 illustrates a block diagram of a system 100 for preventing wrong route/unintentional/unauthorized injection into catheters and connectors for intravascular, hypodermic and neuraxial applications, consistent with one or more exemplary embodiments of the present disclosure. In an exemplary embodiment, system 100 may be connected to a catheter/connector 104. In an exemplary embodiment, catheter/connector 104 may include at least one of an arterial catheter, an epidural catheter, and a venous catheter which all including female end (injection port) or any connected adaptors, stopcock, etc. to these mentioned catheters. In an exemplary embodiment, catheter/connector 104 may further refer to catheters or connectors for intravascular, hypodermic and neuraxial applications, such as stopcocks, adaptors, IV tubing setting, IV catheter connections, epidural catheter connections and injection ports.

In an exemplary embodiment, system 100 may include a female end (connector) 108 connected to a male end (connector) 110. In an exemplary embodiment, system 100 may further include a detection mechanism 112 that may be configured to detect the insertion of an external male end (connector) such as a syringe into female end (connector) 108. In an exemplary embodiment, female end (connector) 108 may provide an injection port, through which an external male end (connector), such as a syringe may be inserted and male end (connector) 110 may be connected to catheter/connector 104.

In an exemplary embodiment, system 100 may further include an input/output (I/O) interface 114 that may be coupled to detection mechanism 112. In an exemplary embodiment, I/O interface 114 may be configured to generate an alarm responsive to the detection of the insertion of an external male end (connector), such as a syringe into female end (connector) 108. In an exemplary embodiment, detection mechanism 112 may further be configured to generate a detection signal responsive to an external male end (connector), such as a syringe being inserted into female end (connector) 108. In an exemplary embodiment, detection mechanism 112 may further be configured to transmit the detection signal to I/O interface 114. In an exemplary embodiment, I/O interface 114 may be configured to generate an alarm responsive to receiving the detection signal. In an exemplary embodiment, the generated alarm may include at least one of a visual, a tactile, and an audio alarm.

In an exemplary embodiment, detection mechanism 112 may include a sensor (switch) that may be disposed at least partially within female end (connector) 108, where the sensor (switch) may be configured to be triggered in response to an external male end (connector), such as a syringe being inserted into female end (connector) 108. In an exemplary embodiment, detection mechanism 112 may include at least one of a mechanical sensor (switch), an electronic sensor (switch), an optical sensor (switch), an ultrasonic sensor (switch), and a magnetic sensor (switch).

In practice, when a healthcare professional inserts an external male end (connector), such as a syringe into female end (connector) 108, detection mechanism 112 may detect the presence of the external male end (connector) within female end (connector) 108 and may generate a detection signal and transmit the generated detection signal to I/O interface 114. Then, I/O interface 114 may generate at least one of a visual, audio or tactile alarm to inform the healthcare professionals that the external male end (connector) is inserted into female end (connector) 108. Such informative alarm may help a healthcare professional to double check the injection port to see if the right port is being used for a particular injection.

In another example, when a patient with suicidal tendencies or a history of substance abuse tries to inject themselves unauthorizedly, detection mechanism 112 may detect the insertion of an external male end (connector), such as a syringe into female end (connector) 108 and may send a detection signal to I/O interface 114. In an exemplary embodiment, I/O interface 114 may generate a local alarm or a central alarm to inform the healthcare professionals of the unauthorized injection. In an exemplary embodiment, a central alarm may refer to an alarm that may be activated in a nursing station. In an exemplary embodiment, I/O interface 114 may further be configured to generate an alarm in an external system, such as a computer, a tablet, or a mobile phone.

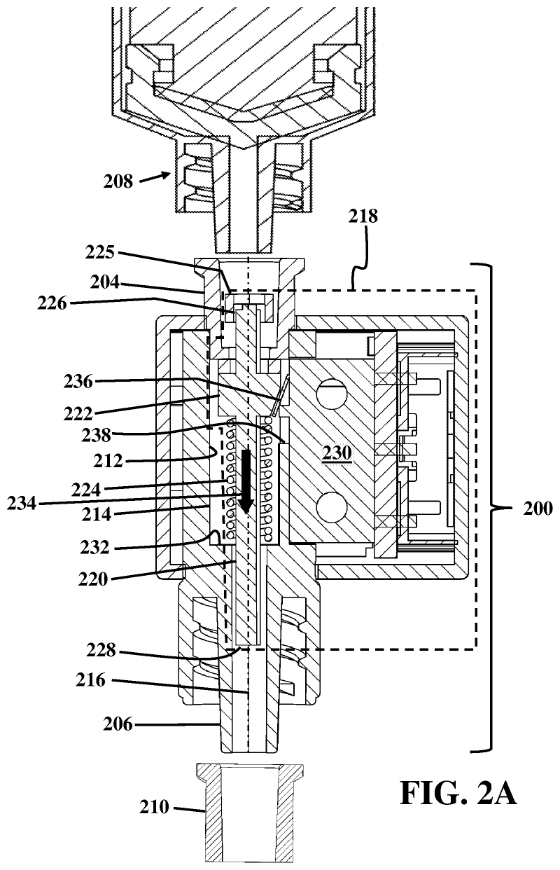

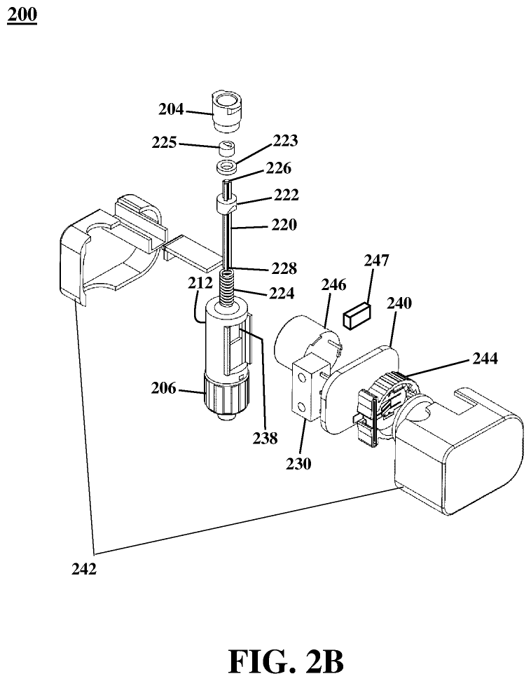

FIG. 2A illustrates a sectional side view of a device 200 for preventing wrong route/unintentional/unauthorized injection into catheters and connectors for intravascular, hypodermic and neuraxial applications, consistent with one or more exemplary embodiments of the present disclosure. FIG. 2B illustrates an exploded view of device 200, consistent with one or more exemplary embodiments of the present disclosure. In an exemplary embodiment, device 200 may be similar to system 100. In an exemplary embodiment, device 200 may be configured to allow for both injecting fluids, such as medications and sampling/aspirating fluids, such as blood.

In an exemplary embodiment, device 200 may include a female end (connector) 204 similar to female end (connector) 108 and a male end (connector) 206 similar to male end (connector) 110. In an exemplary embodiment, female end (connector) 204 may provide an injection port, through which an external male end (connector) 208 may be inserted into female end (connector) 204, and male end (connector) 206 may be connected to a catheter/connector 210. In an exemplary embodiment, catheter/connector 210 may be one of arterial catheter, epidural catheter, venous catheter, and all connected connectors to these catheters. As used herein, catheter/connector 210 may further refer to catheters or connectors for intravascular, hypodermic and neuraxial applications (such as injection ports, stopcocks, adaptors and tubing setting). In an exemplary embodiment, as used herein male/female connectors and male/female ends may be used interchangeably.

In an exemplary embodiment, female end (connector) 204 may include an annular wall 212 encompassing a fluid passage 214 between female end (connector) 204 and male end (connector) 206. In an exemplary embodiment, fluid passage 214 may be extended along a longitudinal axis 216 of fluid passage 214 between female end (connector) 204 and male end (connector) 206.

In an exemplary embodiment, device 200 may further include a detection mechanism 218 similar to detection mechanism 112. In an exemplary embodiment, detection mechanism 218 may be configured to detect the insertion of external male end (connector) 208 into female end (connector) 204. In an exemplary embodiment, detection mechanism 218 may include a mechanical sensor (switch) that may be activated in response to external male end (connector) 208 pressing on the mechanical sensor (switch) 230. What follows is a description of such mechanical sensor (switch) 230 that may be utilized for detecting the presence of an external male end (connector), such as a syringe in an exemplary female end (connector) of the device. However, as mentioned before, the sensor (switch) may be at least one of a mechanical sensor (switch), an electronic sensor (switch), an optical sensor (switch), an ultrasonic sensor (switch), and a magnetic sensor (switch).

In an exemplary embodiment, detection mechanism 218 may include a spring-loaded piston rod 220 that may be coupled to and moveable with a piston 222. In an exemplary embodiment, spring-loaded piston rod 220 may be extended along longitudinal axis 216 of fluid passage 214 between female end (connector) 204 and male end (connector) 206. In an exemplary embodiment, a first end 226 of spring-loaded piston rod 220 may partially extend into female end (connector) 204, and a second opposing end 228 of spring-loaded piston rod 220 may partially extend into male end (connector) 206. In an exemplary embodiment, piston 222 may be mounted on or integrally formed with spring-loaded piston rod 220 between first end 226 and second opposing end 228 of spring-loaded piston rod 220.

In an exemplary embodiment, a washer 223 may be positioned immediately above piston 222. In an exemplary embodiment, washer 223 may be configured to keep the inlet port of device 200 normally closed and the inlet port may be opened only in response to the insertion of an external male end (connector) into female end (connector) 204. Such configuration of washer 223 may allow for preventing blood spillage while sampling blood, washing fluid passage 214, and injecting a medication.

In an exemplary embodiment, spring-loaded piston rod 220 may be loaded with a spring 224 that may be disposed around spring-loaded piston rod 220. In an exemplary embodiment, a conduit of male end (connector) 206 may have a smaller diameter than fluid passage 214, consequently a shoulder 232 may be formed between fluid passage 214 and male end (connector) 206. In an exemplary embodiment, spring 224 may be mounted around spring-loaded piston rod 220 between a lower end of piston 222 and shoulder 232. In an exemplary embodiment, shoulder 232 may provide a stop onto which spring 224 may be compressed in response to piston 222 moving downward toward male end (connector) 206.

In practice, when external male end (connector) 208 is inserted into female end (connector) 204, a tip of external male end (connector) 208 may press against a top surface of first end 226 of spring-loaded piston rod 220, thereby pushing piston 222 downward. In response to the downward movement of piston 222, spring 224 may be compressed. As used herein, a downward movement within fluid passage 214 may refer to a movement along longitudinal axis 216 from female end (connector) 204 towards male end (connector) 206, as shown by arrow 234. In an exemplary embodiment, a piston cap 225 may further be mounted over first end 226 of spring-loaded piston rod 220. In an exemplary embodiment, piston cap 225 may have a through hole that may be configured to allow passage of fluids into fluid passage 214. In other words, in response to external male end (connector) 208 being inserted into female end (connector) 204 and pushing piston 222 downward, the through hole of piston cap 225 may provide fluid communication between external male end (connector) 208 and fluid passage 214.

In an exemplary embodiment, detection mechanism 218 may further include a sensor (switch) 230 that may be mounted between female end (connector) 204 and male end (connector) 206. In an exemplary embodiment, sensor (switch) 230 may include an actuation lever 236 that may be disposed within fluid passage 214. In an exemplary embodiment, annular wall 212 may include a slit 238 that may be configured to allow actuation lever 236 to pass through into fluid passage 214 responsive to sensor (switch) 230 being mounted on an outer surface of annular wall 212 over slit 238. In an exemplary embodiment, sensor (switch) 230 may be mounted over slit 238 in a fluid tight manner. In other words, sensor (switch) 230 may completely seal slit 238 such that no fluid may leak out of fluid passage 214 through slit 238.

In an exemplary embodiment, piston 222 may be configured to trigger sensor (switch) 230 in response to external male end (connector) 208 being inserted into female end (connector) 204. To this end, piston 222 may be in an initial position in the absence of any external male connectors within female end (connector) 204. In an exemplary embodiment, the initial position of piston 222 may correspond to a position immediately above actuation lever 236. In practice, when external male end (connector) 208 is inserted into female end (connector) 204, a tip of external male end (connector) 208 may push spring-loaded piston rod 220 downward in the direction shown by arrow 234, such downward movement of spring-loaded piston rod 220 may urge piston 222 to move downward from the initial position of piston 222 above actuation lever 236, thereby pressing actuation lever 236 and activating sensor (switch) 230.

In an exemplary embodiment, actuation lever 236 of sensor (switch) 230 may function as a mechanical trigger that may be activated in response to piston 222 pressing against actuation lever 236. In an exemplary embodiment, sensor (switch) 230 may be configured to generate a detection signal when actuation lever 236 is pressed by piston 222.

In an exemplary embodiment, sensor (switch) 230 may be coupled to an I/O interface 240 similar to I/O interface 114. In an exemplary embodiment, I/O interface 240 may be mounted in a housing 242 of device 200. In an exemplary embodiment, I/O interface 240 may be configured to generate an alarm responsive to receiving the detection signal generated by sensor (switch) 230. In an exemplary embodiment, the detection signal may include an electric signal generated and transmitted by sensor (switch) 230. Such electric signal may trigger an alarm in I/O interface 240 to alert healthcare professionals that an external male end (connector), such as a syringe is inserted into female end (connector) 204 of device 200 and an injection is about to happen.

In an exemplary embodiment, I/O interface 240 may include an audio signaling device, such as a buzzer 246 that may be configured to generate an audio signal responsive to receiving the detection signal from sensor (switch) 230. In an exemplary embodiment, I/O interface 240 may be connected to a central alarm system via a communication mechanism such as wired or wireless communication modules that may be mounted within housing 242. In an exemplary embodiment, upon reception of a detection signal from sensor (switch) 230, I/O interface 240 may transmit such signal to a remote alarm positioned, for example in a nursing station to generate a central alarm. In an exemplary embodiment, I/O interface 240 may further be connected via wired or wireless links to an external device, such as a computer, a laptop, a tablet, or a cellphone to send alerting messages regarding the insertion of an external male end (connector), such as a syringe into female end (connector) 204 of device 200. In an exemplary embodiment, I/O interface 240 may further include a visual signaling device, such as an LED 247, or a tactile signaling device, such as a vibrator. In an exemplary embodiment, I/O interface 240 may further be configured to generate a visual or tactile signal responsive to receiving the detection signal from sensor (switch) 230.

In an exemplary embodiment, I/O interface 240 may further include a switch/puss-button or any other similar input device for a user to be able to turn the generated visual, tactile, or audio signal off. Alternately, I/O interface 240 may further be configured to generate visual, tactile, or audio signals for a predetermined period of time. Such configuration of I/O interface 240 may allow the healthcare professionals to carry on their tasks, after being informed about the wrong port, without the alarm creating an annoying environment.

In an exemplary embodiment, device 200 including I/O interface 240 may be powered by a battery pack 244 that may be housed within housing 242 or alternatively device 200 may be connected to a power outlet utilizing power cords.

Such configuration of device 200 and how device 200 is equipped with detection mechanism 218 may allow for an easy detection of an external male end (connector), such as a syringe within female end (connector) 204 of device 200. Furthermore, such alarming mechanism provided by device 200 may keep healthcare professionals alert when injecting a substance into female connectors (injection ports) connected to a patient. Even under stressful conditions, an audio, tactile, or visual alarm produced by I/O interface 240 may help caregivers to double check the ports they are about to use for injections. Consequently, the risks associated with a wrong route/unintentional/unauthorized injection into arterial line, epidural line and venous line may be kept at minimum.

In an exemplary embodiment, all electronic components of device 200 that are in contact with fluids, such as sensor (switch) 230 and associated circuits or parts of I/O interface 240 may be insulated or made of nonconductive materials. In an exemplary embodiment, all electronic components of device 200 that are in contact with fluids, such as sensor (switch) 230 and associated circuits or parts of I/O interface 240 may be mounted within device 200 in a fully fluid-tight manner to prevent penetration of fluids into such electronic components.

In an exemplary embodiment, detection mechanism 112 may either be installed within a device for preventing wrong route, unintentional injections into arterial and epidural lines or unauthorized injections into venous, epidural or even arterial lines similar to detection mechanism 218 of device 200, or alternately it may be mounted outside an exemplary device on an exemplary inlet port of an exemplary device.

According to one or more exemplary embodiments, the present disclosure is directed to a method for preventing wrong/unintentional/unauthorized injections. An exemplary method may include providing a device that may include a female end (connector) configured to receive an external male end (connector), and a male end (connector) connected in fluid communication with an exemplary female end (connector), where an exemplary male end (connector) and an exemplary female end (connector) may be configured to form a fluid passage. An exemplary method may further include providing a detecting mechanism for an exemplary device, where an exemplary detection mechanism may be configured to detect the insertion of an exemplary external male end (connector) into an exemplary female end (connector) of an exemplary device. An exemplary method may further include coupling an input/output (I/O) interface to an exemplary detecting mechanism, where an exemplary detecting mechanism may further be configured to urge an exemplary I/O interface to generate an alarm responsive to the detection of the insertion of an exemplary external male end (connector) into an exemplary female end (connector).

In an exemplary embodiment, the step of providing an exemplary detecting mechanism for an exemplary device may refer to either mounting an exemplary detecting mechanism on an exemplary device or an exemplary detecting mechanism being already mounted on an exemplary device. In an exemplary embodiment, the step of coupling an input/output (I/O) interface to an exemplary detecting mechanism may refer to either coupling an exemplary input/output (I/O) interface to an exemplary detecting mechanism or an exemplary input/output (I/O) interface being already coupled to an exemplary detecting mechanism.

In an exemplary embodiment, the step of providing an exemplary detecting mechanism for an exemplary device may refer to mounting an exemplary detecting mechanism within an exemplary female end (connector) of an exemplary device or within an exemplary fluid passage formed by an exemplary female end (connector) and an exemplary male end (connector) of an exemplary device. In an exemplary embodiment, the step of providing an exemplary detecting mechanism for an exemplary device may alternately refer to mounting an exemplary detecting mechanism outside an exemplary device on an exemplary female end (connector) of an exemplary device.

According to one or more exemplary embodiments, the present disclosure is directed to a method for preventing wrong/unintentional/unauthorized injections. An exemplary method may include detecting the insertion of an external male end or end (connector) into a female end (connector) coupled to intravascular, hypodermic and neuraxial catheters or connectors, generating an alarm in response to the detection of the insertion of an exemplary external male end or end (connector) into an exemplary female end (connector).

An exemplary step of detecting the insertion of an external male end (connector) into a female end (connector) may include coupling a sensor with an exemplary female end (connector) or an exemplary fluid passage providing a fluid communication between an exemplary female end (connector) and arterial/venous/epidural catheters or connectors. An exemplary sensor may be configured to detect the insertion of an exemplary external male end (connector) into an exemplary female end (connector).

An exemplary step of generating an alarm may include generating an audio/visual/tactile signal in response to the detection of the insertion of an exemplary external male end or into an exemplary female end (connector). An exemplary step of generating an alarm may include coupling an input/output (I/O) interface to an exemplary sensor and then configuring an exemplary I/O interface to generate an alarm responsive to the detection of the insertion of an exemplary external male end (connector) into an exemplary female end (connector).

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for description and not of limitation, such that the terminology or phraseology of the present specification is to be interpreted by the skilled artisan in light of the teachings and guidance.

The breadth and scope of the present disclosure should not be limited by any of the above-described exemplary embodiments but should be defined only in accordance with the following claims and their equivalents.

Throughout this specification and the claims which follow, unless the context requires otherwise, the word “comprise”, and variations such as “comprises” or “comprising”, will be understood to imply the inclusion of a stated integer or step or group of integers or steps but not to the exclusion of any other integer or step or group of integers or steps.

Moreover, the word “substantially” when used with an adjective or adverb is intended to enhance the scope of the particular characteristic; e.g., substantially planar is intended to mean planar, nearly planar and/or exhibiting characteristics associated with a planar element. Further use of relative terms such as “vertical”, “horizontal”, “up”, “down”, and “side-to-side” are used in a relative sense to the normal orientation of the apparatus.

What is claimed is:

1. A device for preventing wrong/unintentional/unauthorized injections, the device comprising:
a female end configured to receive an external male connector;
a male end connected in fluid communication with the female end, the male end and the female end configured to form a fluid passage;
a detecting mechanism configured to detect the insertion of the external male connector into the female end; and
an input/output (I/O) interface coupled to the detecting mechanism, the detecting mechanism further configured to urge the I/O interface to generate an alarm responsive to the detection of the insertion of the external male connector into the female end.

2. The device of claim 1, wherein the alarm comprises at least one of a local alarm and a central alarm.

3. The device of claim 2, the detection mechanism is further configured to generate a detection signal, the I/O interface configured to generate the alarm responsive to receiving the detection signal from the detection mechanism.

4. The device of claim 3, wherein the alarm comprises at least one of an audio signal, a visual signal, and a tactile signal.

5. The device of claim 4, wherein the detecting mechanism comprises a sensor configured to be triggered in response to the external male connector being inserted into the female end.

6. The device of claim 5, wherein the sensor comprises at least one of a mechanical sensor, an electronic sensor, an optical sensor, an ultrasonic sensor, and a magnetic sensor.

7. The device of claim 5, wherein the detection mechanism comprises:
a spring-loaded piston rod, the spring-loaded piston rod extended along a longitudinal axis of the fluid passage, the spring-loaded piston rod configured to be moveable along the longitudinal axis of the fluid passage; and
a piston coupled to and moveable with the spring-loaded piston rod; and
a sensor coupled with the female end, the piston configured to trigger the sensor in response to the external male connector being inserted into the female end.

8. The device of claim 7, wherein spring-loaded piston rod is extended along the longitudinal axis of the fluid passage, the spring-loaded piston rod extended between a first end disposed within the female end and an opposing second end disposed within male end.

9. The device of claim 8, wherein the first end is configured to provide a contact surface below the injection port, the spring-loaded piston rod configured to move along the longitudinal axis of the fluid passage towards the male end in response to the external male connector being inserted into the female end pressing against the contact surface.

10. The device of claim 9, wherein the piston is configured to contact the sensor in response to the spring-loaded piston rod moving along the longitudinal axis of the fluid passage towards the male end, the sensor configured to be activated responsive to the contact with the piston.

11. The device of claim 10, wherein the sensor comprises a switch with a mechanical trigger, the mechanical trigger position within the female end, the piston configured to contact and press the mechanical trigger in response to the spring-loaded piston rod moving along the longitudinal axis of the fluid passage towards the male end, the switch configured to generate a detection signal responsive to the mechanical trigger being pressed.

12. The device of claim 11, wherein the female end further comprises an annular wall encompassing a fluid passage between the female end and the male end, the annular wall comprising a slit extended along the longitudinal axis of the female end, the switch mounted on the slit in a fluid-tight manner, the mechanical trigger of the switch comprising an actuation lever extended into the female end through the slit.

13. The device of claim 11, wherein the piston is configured to contact and press the actuation lever in response to the spring-loaded piston rod moving along the longitudinal axis of the fluid passage towards the male end.

14. A device for preventing unintentional/wrong/unauthorized injections, the device comprising:
a female end configured to receive an external male connector;
a male end connected in fluid communication with the female end, the female end further comprising an annular wall encompassing a fluid passage between the female end and the male end;
a switch with an actuation lever mounted on the annular wall, the annular wall comprising a slit, the actuation lever configured to extend into the female end via the slit in a fluid-tight manner;
a spring-loaded piston rod coaxially mounted within the fluid passage;
a piston coupled to the spring-loaded piston rod, the piston moveable along the longitudinal axis of the fluid passage with the spring-loaded piston rod,
wherein, the spring-loaded piston rod is extended along the longitudinal axis of the fluid passage between a first end disposed within the female end and an opposing second end disposed within the male end, the first end configured to provide a contact surface within the female end, the spring-loaded piston rod configured to move along the longitudinal axis of the fluid passage towards the male end in response to the external male connector being inserted into the female end pressing against the contact surface,
wherein, the piston is configured to contact and press the actuation lever in response to the spring-loaded piston rod moving along the longitudinal axis of the fluid passage towards the male end, the switch configured to generate a detection signal responsive to the mechanical trigger being pressed.

15. The device of claim 14, further comprising an input/output (I/O) interface coupled to the switch, the switch further configured to transmit the detection signal to the I/O interface, the I/O interface configured to generate an alarm responsive to receiving the detection signal.

16. The device of claim 15, wherein the alarm comprises at least one of a local alarm and a central alarm.

17. The device of claim 16, wherein the alarm comprises at least one of an audio signal, a tactile signal, and a visual signal.

18. The device of claim 16, wherein the alarm is further configured to be selectively turned off.

19. The device of claim 14, wherein the male end is configured to be connected to catheters and connectors for intravascular, hypodermic and neuraxial applications, the catheters and connectors comprising at least one of arterial, epidural and venous catheters and all connected connectors to arterial, epidural and venous lines.

20. The device of claim 14, wherein the male end is further configured to be compatible with all types of female ends and small-bore connectors for intravascular, hypodermic and neuraxial applications.

21. The device of claim 20, wherein the female end is configured to be compatible with all types of male connectors and small-bore connectors for intravascular, hypodermic and neuraxial applications.
The device of claim 14, wherein the male end and the female end are configured to be compatible with all types of female/male end and small-bore connectors used in intravascular, hypodermic and neuraxial applications, the female/male end and small-bore connectors comprising at least one of lock connectors, Luer lock connectors, slip connectors, Luer slip connectors, syringes, stopcocks.

22. The device of claim 14, further comprising a washer mounted immediately above the piston, the washer configured to prevent blood spillage during injection and sampling procedures.

23. A method for preventing wrong/unintentional/unauthorized injections, the method comprising:
providing a device, the device comprising:
a female end configured to receive an external male connector; and
a male end connected in fluid communication with the female end, the female end further comprising an annular wall encompassing a fluid passage between the female end and the male end;
detecting the insertion of an external male connector into the female end; and
generating an alarm in response to the detection of the insertion of the external male connector into the female end.

24. The method according to claim 24, wherein detecting the insertion of an external male connector into the female end comprises associating a detection mechanism with the female end, the detection mechanism configured to detect the insertion of the external male connector into the female end.

25. The method according to claim 25, wherein generating an alarm comprises generating an audio/visual/tactile signal in response to the detection of the insertion of an external male connector into the female end.

26. The method according to claim 26, generating an alarm comprises configuring an exemplary I/O interface coupled to the detection mechanism to generate an alarm responsive to the detection of the insertion of an external male connector into the female end.
